(19) United States
(12) Patent Application Publication
(10) Pub. No.: US 2014/0012322 A1
(43) Pub. Date: Jan. 9, 2014

(54) Bone Screw

(71) Applicants: Brian Gayvey, Landenberg, PA (US); Mark Fromhold, Ardmore, PA (US)

(72) Inventors: Brian Gayvey, Landenberg, PA (US); Mark Fromhold, Ardmore, PA (US)

(21) Appl. No.: 13/792,439

(22) Filed: Mar. 11, 2013

Related U.S. Application Data
(63) Continuation-in-part of application No. 12/249,526, filed on Oct. 10, 2008.

(51) Int. Cl. A61B 17/70

(52) U.S. Cl. CPC A61B 17/7071
USPC 606/279

(57) ABSTRACT

The present invention provides a bone screw which can be used for fixation and/or fastening of prosthetic devices or instruments to bone tissue whose structure or dimensions differ from one region to another. In particular, the present invention provides a bone screw which is designed to optimize purchase in both the cancellous and cortical regions of a vertebral body. In an exemplary embodiment, the bone screw has a distal portion and a proximal portion in which the diameter of the thread on the proximal portion of the screw is greater than the diameter of the thread on the distal portion of the screw.

### CROSS REFERENCE TO RELATED APPLICATIONS

This patent application is a continuation-in-part application of U.S. patent application Ser. No. 12/249,526, filed Oct. 10, 2008, the entire contents of which are incorporated by reference.

### FIELD OF THE INVENTION

The present invention relates to the field of orthopedic surgery and more specifically to a bone screw for orthopedic use.

### BACKGROUND OF THE INVENTION

As is known in the field of orthopedic surgery, and more specifically spinal surgery, bone screws may be used for fixation or for the fastening of prosthetic devices or instruments to bone tissue. An exemplary use of bone screws may include using bone screws to fasten a prosthetic device, such as a bone plate or a spinal spacer, to a vertebral body for the treatment of a defect in a patient's spine, such as a fracture within a vertebral body or a degenerating intervertebral disc. Focusing on the bone plate example, bone screws can be used to fasten anchors to a number of vertebral bodies and a bone plate can then be connected to the vertebral bodies using the anchors to fuse a segment of the spine. In another exemplary use, bone screws can be used to fix the location of a spinal spacer once the spacer is implanted between adjacent vertebral bodies.

The bone tissue that comprises the vertebral body, in terms of mechanical characteristics, can be divided into two distinct regions, namely, cancellous bone tissue, which is characterized by voids and a low density, and cortical bone tissue, which is a higher density, stronger bone region. Since the cortical bone tissue region is stronger than the cancellous bone tissue, the cortical bone tissue is better able to support a secure connection for screw fixation than the cancellous bone tissue.

As such, there exists a need for a bone screw that is able to optimally purchase bone tissue where the mechanical characteristics of the bone tissue vary from one region to another to improve fixation and/or fastening of prosthetic devices or instruments to bone tissue.

### SUMMARY OF THE INVENTION

The present invention provides a bone screw which can be used for fixation and/or fastening of prosthetic devices or instruments to bone tissue whose structure or dimensions differ from one region to another. In particular, the present invention provides a bone screw which is designed to optimize purchase in both the cancellous and cortical regions of the vertebral body. In an exemplary embodiment, the bone screw has a distal portion and a proximal portion, each portion having an approximately constant diameter over a portion of its length, in which the diameter of the thread on the proximal portion of the screw is greater than the diameter of the thread on the distal portion of the screw.

Further areas of applicability of the present invention will become apparent from the detailed description provided hereinafter. It should be understood that the detailed description and specific examples, while indicating the preferred or exemplary embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of an exemplary embodiment of the bone screw according to the present invention;

FIG. 2 is a side perspective view of the bone screw shown in FIG. 1;

FIG. 3 is an enlarged partial cross-sectional view of the bone screw shown in FIG. 1;

FIG. 4 is an enlarged partial cross-sectional second side view of the bone screw shown in FIG. 1;

FIG. 5 is a side view of an alternative bone screw according to the present invention; and

FIG. 6 is a top view of a novel trajectory of the alternative bone screw of FIG. 5.

### DETAILED DESCRIPTION OF THE EMBODIMENTS

The following description of the preferred embodiment(s) is merely exemplary in nature and is in no way intended to limit the invention, its application, or uses.

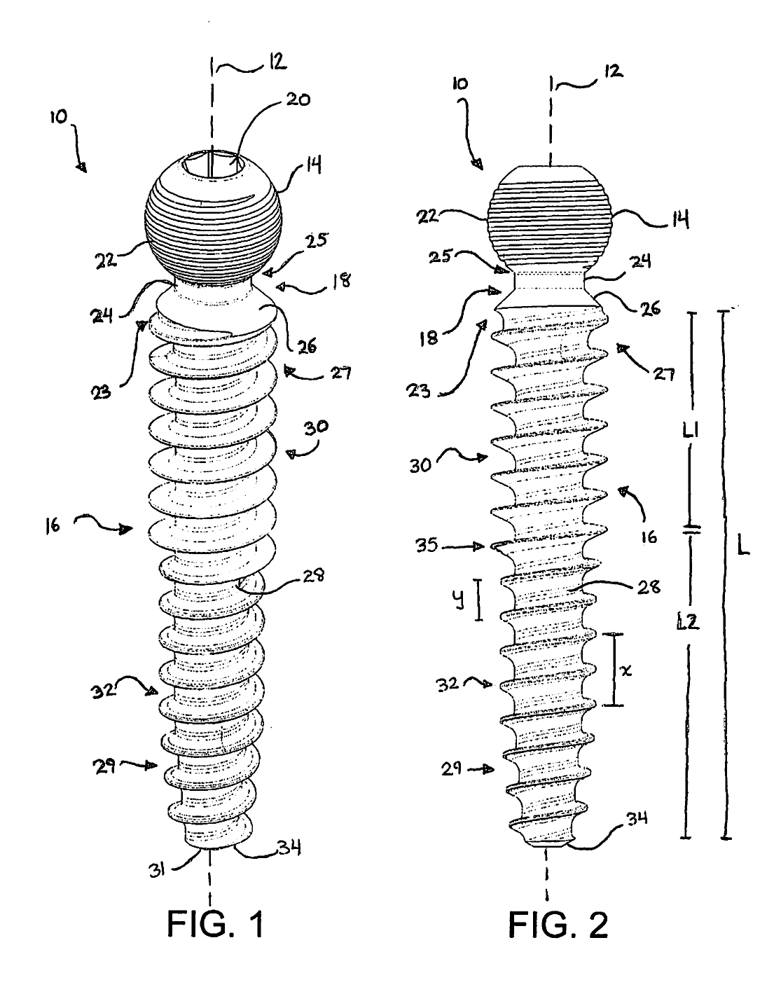

With reference to FIGS. 1 and 2, a preferred embodiment of a bone screw 10 according to the present invention is illustrated. The bone screw 10 preferably includes, concentric to a longitudinal axis 12, a head portion 14, a neck portion 18 and a shank portion 16. The head portion 14 connects to the shank portion 16 through the neck portion 18. The bone screw 10 is preferably constructed from any biocompatible material including, but not limited to, stainless steel alloys, titanium, titanium based alloys, or polymeric materials.

In a preferred embodiment, the head portion 14 of bone screw 10 has a generally spherical shape and includes a recess 20 for receiving a driving instrument. As is well known in the art, the recess 20 may be configured and dimensioned to any shape that corresponds with the end of the driving instrument designed to engage the bone screw 10. For example, the recess 20 may be any one of the following shapes: slot, cross, polygon, or multi-lobes. The generally spherical shape of the head portion 14 is configured and dimensioned to be received within a correspondingly shaped cavity in a receiving member (not shown) which may be part of a spinal fixation system. The shape of the head portion 14 allows the bone screw 10 to pivot, rotate and/or move with respect to the receiving member. In an exemplary use, the head portion 14 of the bone screw 10 is received in the cavity of the receiving member and the bone screw 10 is pivoted, rotated or moved until the desired orientation with respect to the receiving member is met. The bone screw 10 is then locked in place in the cavity of the receiving member. In a further preferred embodiment, the head portion 14 also includes texturing 22 that extends along at least a portion of the head portion 14. The texturing 22 on the head portion 14 provides additional frictional surfaces which aid in locking the bone screw 10 in place with respect to the receiving member.

With continued reference to FIGS. 1 and 2, in a preferred embodiment, the neck portion 18 of the bone screw 10 integrally connects the head portion 14 with the shank portion 16. Preferably, the neck portion 18 includes a generally cylindrical region 24 and a truncated generally frustoconical region 26. The diameter of the distal end 23 of the frustoconical region 26 is preferably dimensioned to match a major diameter (discussed below) of the bone screw 10 while the diameter of the proximal end 25 of the frustoconical region 26 is preferably dimensioned to match the diameter of the generally cylindrical region 24 of the neck portion 18. In a preferred embodiment, the generally cylindrical region 24 will have a diameter that is at least as large as a minor diameter (discussed below) of the bone screw 10, but the diameter of the generally cylindrical region 24 can be smaller than the minor diameter of the bone screw 10. By having the diameter of the neck portion 18 dimensioned at least as large as a minor diameter of the bone screw 10, the overall rigidity and strength of the bone screw 10 is increased.

Turning to FIGS. 1-4, in a preferred embodiment, the shank portion 16 of the bone screw 10 includes a shaft 28, having a length L, surrounded at least in part by a plurality of thread portions 30, 32. The diameter of the shaft 28 is the minor diameter of the bone screw 10 and the diameter of the shaft 28 including the thread portions 30, 32 is the major diameter of the screw 10. In a preferred embodiment, the diameter of the shaft 28 remains generally constant from the proximal end 27 toward the distal end 29 of the shaft 28. However, the diameter of the distal end 29 of the shaft 28 preferably decreases towards the distal tip 34 of the bone screw 10. The constant diameter of a majority portion of the shaft 28 allows for optimal screw positioning when the bone screw is inserted into a predetermined area in the bone tissue. The constant diameter also allows for varying the depth positioning of the bone screw in the bone. For example, if a surgeon places the bone screw 10 into bone tissue at a first depth and decides the placement is more optimal at a second, shallower depth, the bone screw 10 can be backed out to the second depth and still remain fixed in the bone. In another embodiment, the diameter of the shaft 28 may vary along its length, including increasing in diameter from the proximal end to the distal end or decreasing in diameter from the proximal end to the distal end.

Looking at FIGS. 1-2, the plurality of threads 30, 32 surrounding the shaft 28 extend, in a preferred embodiment, from the distal tip 34 of the shaft 28 to the distal end 23 of the frustoconcial region 26 of the neck portion 18. In another preferred embodiment, the threads 30, 32 may extend along only a portion of shaft 28. As seen in FIGS. 1-2, the thread portions 30, 32 are preferably a Modified Buttress thread but the threads can be any other type of threading that is anatomically conforming, including, but not limited to Buttress, Acme, Unified, Whitworth and B&S Worm threads.

In a preferred embodiment, the diameter or depth or height (hereinafter, diameter) of the thread portion 30 remains substantially constant over its length L1 and the diameter of the thread portion 32 remains substantially constant over a portion of its length L2. Preferably, the diameter of the thread portion 32 decreases towards the distal tip 34 of the bone screw 10. By having a decreased diameter thread portion 32 near the distal tip 34 of the bone screw 10, the bone screw 10 can be self-starting. In another preferred embodiment, bone screw 10 may also include at least one flute to clear any chips, dust, or debris generated when the bone screw 10 is implanted into bone tissue.

In a preferred embodiment, the thread portion 30 also differs dimensionally from the thread portion 32. More specifically, the thread portion 30 preferably has a larger diameter than the thread portion 32. The diameter of the thread portion is determined by subtracting the minor diameter from the major diameter of the bone screw 10. For example, if the minor diameter of the bone screw 10 is 4 mm and the major diameter of the bone screw 10 near the proximal end 27 of the shaft 16 is 7 mm, the diameter of the thread portion 30 around the proximal end 27 of the shaft 16 is 3 mm. A preferred difference in the diameter between the thread portion 30 and the thread portion 32 is 2.0 mm but a larger or smaller difference between the thread portion diameters is also contemplated. In a preferred embodiment, the ratio of the diameter of the thread portion 30 to the ratio of the thread portion 32 is approximately 1.2, but can vary from 1.0 to 1.5

By having a larger diameter thread portion 30 and a smaller diameter thread portion 32, the bone screw 10 can grip bone tissue having regions with varying mechanical characteristics in an optimal manner. The larger diameter thread portion 30, which surrounds the proximal portion 27 of the shaft 16, is better suited to grip the cancellous region of the bone. The larger diameter thread portion, having the larger threads and increased purchasing surface area, better engages the softer, less dense bone tissue. Correspondingly, the smaller diameter thread portion 32, which surrounds the distal portion 29 of the shaft 16, is better suited to grip the cortical region of the bone. Since the cortical region is harder and denser, a smaller thread is preferred for the bone screw 10 to optimally purchase that bone tissue region. The combination of the larger diameter thread portion 30 and the smaller diameter thread portion 32 provides for an improved bone screw having greater bone tissue purchasing as well as greater pull-out strength than a screw with a single diameter thread. In a preferred embodiment, the improved purchasing lowers the bone screw 10 toggling over time and the pull out-strength of bone screw 10 compared to a screw having a single outer diameter thread has been determined to be at least 20% higher than the pull-out strength of the screw having a single outer diameter thread.

In a preferred embodiment, the bone screw 10 also has a transition portion 35 between thread portions 30, 32 to allow for easier insertion of the bone screw 10 in the bone tissue. Preferably, over the transition portion 35, the major diameter of the bone screw 10 decreases gradually between the thread portion 30 and thread portion 32 when viewed from a proximal to distal direction.

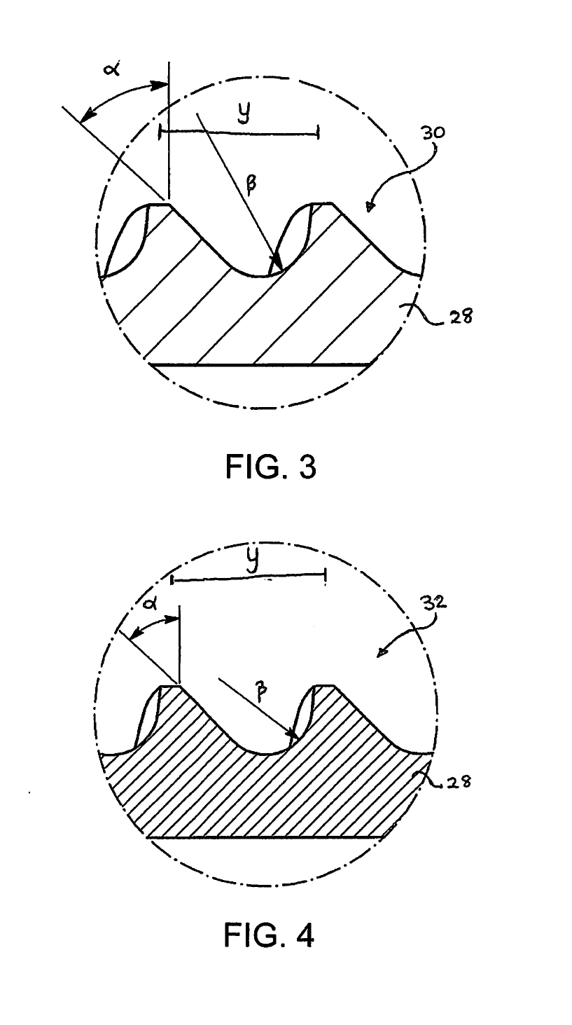

Looking now at FIGS. 3-4, enlarged cross-sectional views of the thread portions 30, 32 can be seen. In a preferred embodiment, the thread angle α of the thread portions 30, 32 is preferably 25°, but can be between 20°-30°. It has been determined that this range of thread angles is optimal for purchasing in the different regions of the bone tissue. The radius 13 of the thread portion 30, 32 is preferably 0.5 mm, but can be between 0.1 mm-1 mm. Again, it has been determined that this range for the radius is optimal for purchasing in the different regions of the bone tissue.

Turning back to FIGS. 1-4, the thread portions 30, 32 on the shaft 28 of the screw 10 is preferably a multi-start thread. More specifically, in a preferred embodiment, thread portions 30, 32 of the bone screw 10 is a two-start thread. Multi-start threads have the advantage of providing a thread on a screw shaft that has a smaller thread pitch (discussed below) than would be the case if the thread is a single-start thread. A smaller thread pitch can enhance the security of the fixation in bone tissue as well as increase the rate of installation of the screw in the bone tissue. In other preferred embodiments, a single-start thread portion as well as three or more start thread portion is also contemplated.

As mentioned above, the thread pitch is defined as the distance along the axis of the screw between adjacent thread peaks, shown in FIG. 2 as y. The thread lead is defined as the distance that is traveled along the axis of the screw in one complete 360° revolution of the screw, shown in FIG. 2 as x. In a preferred embodiment, the number of starts of the thread portions 30, 32 is equal to the ratio of the thread lead x to the thread pitch y. For example, for a two-start thread, the thread lead is preferably 5 mm and the thread pitch is preferably 2.5 mm. In a preferred embodiment, the bone screw 10 includes a two start thread. In another preferred embodiment, the thread pitch y is substantially constant over thread portions 30, 32.

Dimensions, in millimeters, of a preferred embodiment of the bone screw 10, which is suitable for use as a bone screw in a vertebral body, are as follows:

| L | L1 | L2 |
|---|---|---|
| 30.0 | 15.0 | 15.0 |
| 35.0 | 20.0 | 15.0 |
| 40.0 | 20.0 | 20.0 |
| 45.0 | 25.0 | 20.0 |
| 50.0 | 25.0 | 25.0 |
| 55.0 | 30.0 | 25.0 |
| 60.0 | 35.0 | 25.0 |
| 65.0 | 40.0 | 25.0 |
| 70.0 | 45.0 | 25.0 |
| 75.0 | 50.0 | 25.0 |
| 80.0 | 55.0 | 25.0 |
| 85.0 | 60.0 | 25.0 |
| 90.0 | 65.0 | 25.0 |
| 95.0 | 70.0 | 25.0 |
| 100.0 | 75.0 | 25.0 |

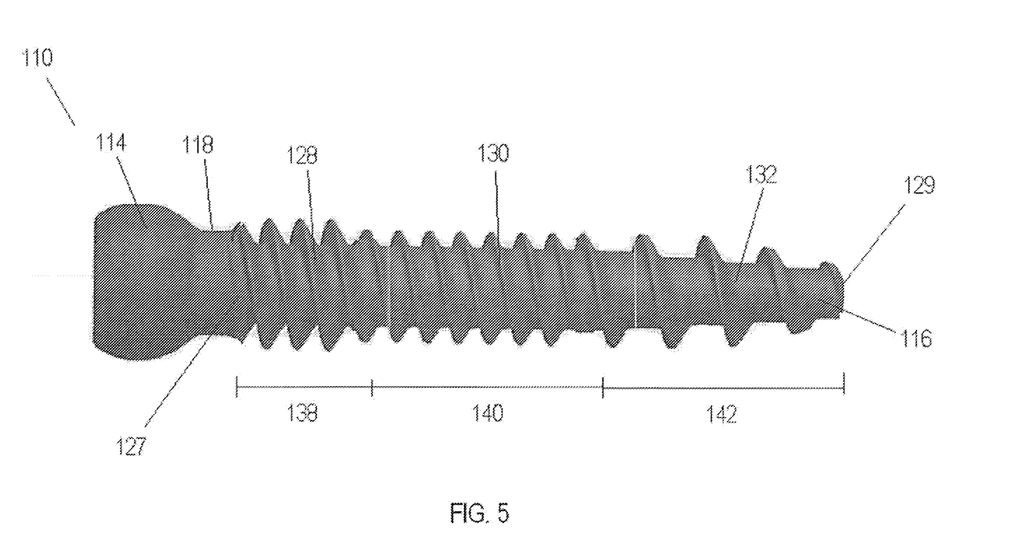

FIG. 5 is a side view of an alternative bone screw according to the present invention. The bone screw 110 in FIG. 5 can be used in many different types of bone, but is particularly advantageous as a type of cortical pedicle screw. The bone screw 110 includes a shank portion 116 with three distinct, threaded sections: a proximal portion 138, an intermediate portion 140 and a distal portion 142. The distal portion 142 includes novel features that allow it to accommodate cancellous (e.g., trabecular) bone. The intermediate portion 140 includes novel features that allow it to accommodate stronger cortical bone. The proximal portion 138 includes novel features that allow it to accommodate very hard and dense cortical bone. These features, as well as a novel trajectory made possible by these features, will be described in more detail below.

The bone screw 110 includes a head portion 114, a neck portion 118 and a shank portion 116. The head portion 114 can include a number of the features described above, including a generally spherical shape and a recess for receiving a driving instrument. In some embodiments, the recess can be any of the following shapes: slot, cross, polygon or multi-lobes. The generally spherical shape of the head portion 14 is configured and dimensioned to be received within a correspondingly shaped cavity in a receiving member (not shown) which may be part of a spinal fixation system. The shape of the head portion 114 allows the bone screw 110 to pivot, rotate and/or move with respect to the receiving member. In some embodiments, the head portion 114 also includes texturing that extends along at least a portion of the head portion 114. The texturing on the head portion 114 provides additional frictional surfaces which aid in locking the bone screw 110 in place with respect to the receiving member.

The head portion 114 transitions into the neck portion 118, which comprises a substantially cylindrical member that extends in between the head portion 114 and the shank portion 116. In alternative embodiments, the neck portion 118 can further include a truncated generally frustoconical region (as in FIG. 2) that transitions directly into the threaded shank portion 116. In some embodiments, the neck portion 118 comprises a cylindrical region having a diameter that is larger than a largest diameter of the shank portion 116, thereby advantageously increasing the overall rigidity and strength of the bone screw 110.

The neck portion 118 transitions into the shank portion 116, which comprises three distinct sections not seen in prior bone screws: a proximal portion 138, an intermediate portion 140 and a distal portion 142. As the distal portion 142 comprises the leading end of the bone screw (e.g., the end that will first enter into a bone member), this will be the first section described in more detail herein. In the present embodiment, each of the three sections of the shank portion 116 includes one or more threads. In other embodiments, one or more sections can be absent of threads. For example, the proximal portion 138 and the distal portion 142 can bear threads, whereas the intermediate section 140 can be absent of threads. In addition, in the present embodiment, the shank portion 116 remains substantially constant in diameter along a majority of the length of the shank portion 116, from a proximal end 127 of the shank portion 116 to a distal end 129 of the shank portion, with a slight taper in the distal portion 142. In other embodiments, the diameter of the shank portion 116 can vary, such that it increases or decreases along different portions of the shank portion.

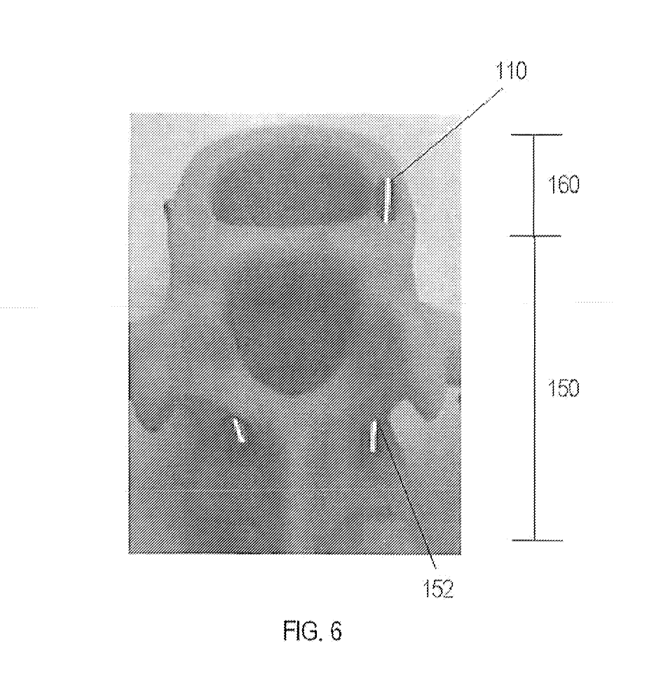

In some embodiments, the distal portion 142 of the shank portion 116 comprises a tapered distal end to assist in entry into a bone member. In some embodiments, the distal portion 142 comprises a threaded portion 132 including a single-lead thread with a wide pitch (e.g., such as between 3 and 6 mm, or greater than about 4 mm). While in some embodiments, each of the sections 128, 130, 132 of the bone screw 110 can encounter and accommodate cortical bone (as shown in FIG. 6), the distal portion 142 of the bone screw 110 is the most likely to also encounter cancellous (e.g., trabecular) bone, which is less dense than cortical bone. As such, the wide pitch advantageously allows for more of the weaker cancellous bone to be located within the threads themselves, thereby providing increased resistance to pullout or migration.

In some embodiments, the intermediate portion 140 of the shank portion 116 comprises a substantially constant diameter. In some embodiments, the intermediate portion 140 comprises a threaded portion 130 including a dual-lead thread, made with a pitch that is approximately half that of the wide pitch in the distal portion 142. In some embodiments, the dual-lead threads will be completely separate from the single-lead thread in the distal portion 142 of the screw. However, in the present embodiment, one of the threads of the dual-lead thread will be a continuation of the single-lead thread in the distal portion 142 of the screw. This way, as the screw 110 advances through bone, one lead in the intermediate portion 140 will follow the thread tract of the distal portion 142, and the second lead of the intermediate portion 140 will cut a new thread tract in approximately the middle of the distal thread tract. In some embodiments, the intermediate portion 140 of the shank portion 116 will be located in a region of strong cortical bone, and the dual-lead threads will advantageously increase the purchase in bone in this region. In some embodiments, a diameter of the outer, major threads in the intermediate portion 140 substantially matches a diameter of the outer, major threads in the distal portion 142.

In some embodiments, the proximal portion 138 of the shank portion 116 comprises a substantially constant diameter. In some embodiments, the proximal portion 138 comprises a threaded portion 128 including a dual-lead thread made with similar leads and pitch as the threaded portion 130 in the intermediate portion 140. While the leads and pitch of the dual-lead thread of the proximal portion 138 can be similar to, if not identical, to the dual-lead thread in the intermediate portion 140, the threads in the proximal portion 138 of the shank portion 116 contain a larger thread cross-section diameter having increased diameter compared to the threads in the intermediate portion 140 of the shank portion 116. For example, in some embodiments, the thread cross-section diameter in the distal portion 142 and/or immediate portion 140 of the screw can have a diameter of between 4.0-10.5 mm, while the thread cross-section diameter in the proximal portion 138 can have a diameter of between 5.0-11.5 mm. In some embodiments, the proximal portion 138 can have a thread cross-section diameter that is at least 1 mm larger than a thread cross-section diameter in the distal portion 142 and/or immediate portion 140 of the screw 110. This larger diameter of the threads in the proximal portion 138 advantageously accommodates cortical bone that is very hard and dense.

Using the novel bone screw 110 described above, a distinct trajectory can be carved out through bone. FIG. 6 is a top view of a novel trajectory of the alternative bone screw 110. This trajectory is discussed in further detail below.

With reference to FIG. 6, the vertebra can be viewed as two separate sections, the vertebral elements 150 (e.g., facets, pars, processes) and the vertebral body 160. Advantageously, the bone screw 110 described above is capable of an approach whereby the screw is inserted through the lateral aspect of the pars in-line with the inferior border of the pedicle. As shown in FIG. 6, the features of the bone screw 110 allow the bone screw to be inserted primarily through dense, cortical bone, and extend all the way into the vertebral body 160 of the vertebra. As shown in FIG. 6, the distal end (e.g., the leading end) of the bone screw 110 clearly extends into the vertebral body, which can include both cortical and cancellous bone. As this is the end that is most likely to encounter cancellous bone, the distal end of the bone screw 110 has (as discussed above) a wider pitch to accommodate this type of bone. Advantageously, the trajectory provided by this screw 110 engages more dense cortical bone than alternative pedicle screw trajectories, thereby providing increased fixation, especially in osteoporotic patients. In addition, the trajectory advantageously requires less retraction than alternative pedicle screw trajectories, which results in less morbidity in patients requiring surgery, as well as minimum scarring. Furthermore, the trajectory has minimal risk to spinal cord injury.

While the trajectory described herein (e.g., starting at the lateral aspect of the pars in-line with the inferior border of the pedicle and extending into the vertebral body) is a unique and novel trajectory made possible by the bone screw's unique features, alternative trajectories are also possible. For example, in some embodiments, the bone screw 110 has starting points on the pars, lamina or inferior region of a superior facet and traverses straight ahead or lateral and enters into the posterior vertebral body. In other embodiments, the bone screw 110 can have an approach whose starting point is upslope of the transverse process in a north-south direction, and continues in-line with the lower aspect of an inferior articulating facet. In yet other embodiments, the bone screw 110 can have an approach whose starting point is a couple millimeters medial to a lateral aspect of the pars in-line with an inferior border of the pedicle.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method of inserting a bone screw comprising:
providing a bone screw for insertion into a vertebra, the vertebra comprising a vertebral body and vertebral elements;
inserting the bone screw through a pars of the vertebral elements; and
extending the bone screw into the vertebral body,
wherein the bone screw comprises a head portion, a neck portion and a shank portion, wherein the shank portion includes a proximal portion, an intermediate portion and a distal portion, wherein the distal portion comprises a single-lead thread and the proximal and intermediate portions comprise a dual-lead thread, wherein a pitch of the single-lead thread in the distal portion is greater than a pitch of the dual-lead thread in the proximal and intermediate portions, and wherein the proximal portion has a greater thread cross-section diameter than the intermediate and distal portions.

2. The method of claim 1, wherein the shank portion of the bone screw is substantially constant along a majority of an entire length of the shank portion.

3. The method of claim 1, wherein the distal portion of the shank portion includes a slight taper.

4. The method of claim 1, wherein the single-lead thread in the distal portion of the shank portion continuously forms one of the threads of the dual-lead thread in the intermediate portion of the shank.

5. The method of claim 1, wherein the pitch of the single-lead thread in the distal portion is between 3-6 mm.

6. The method of claim 1, wherein the pitch of the single-lead thread in the distal portion is greater than 4 mm.

7. The method of claim 1, wherein a greatest thread cross-section diameter of the proximal portion of the shank portion is at least 1 mm greater than a greatest thread cross-section diameter of the intermediate portion of the shank portion.

8. A method of inserting a bone screw comprising:
providing a bone screw for insertion into a vertebra, the vertebra comprising a vertebral body and vertebral elements;
inserting the bone screw through a pars of the vertebral elements; and
extending the bone screw into the vertebral body,
wherein the bone screw comprises a head portion, a neck portion and a shank portion, wherein the shank portion includes a proximal portion, an intermediate portion and a distal portion, wherein the distal portion comprises a single-lead thread and the proximal and intermediate portions comprise a dual-lead thread.

9. The method of claim 8, wherein the dual-lead thread of the proximal and intermediate portions has a pitch approximately half the size of a pitch of the single-lead thread of the proximal portion.

10. The method of claim 8, wherein the shank portion has a slight taper along a majority of its length from a proximal end to a distal end.

11. The method of claim 8, wherein extending the bone screw into the vertebral body comprises extending the bone screw into cancellous bone.

12. The method of claim 8, wherein extending the bone screw into the vertebral body comprises extending the bone screw into cortical bone only.

13. The method of claim 8, wherein the intermediate portion has a thread pitch that is approximately half the thread pitch of the distal portion.

14. A method of inserting a bone screw comprising:
providing a bone screw for insertion into a vertebra, the vertebra comprising a vertebral body and vertebral elements; and
inserting the bone screw through a pars of the vertebral elements,
wherein the bone screw comprises a head portion, a neck portion and a shank portion, wherein the shank portion includes a proximal portion, an intermediate portion and a distal portion, wherein the distal portion comprises a single-lead thread and the proximal and intermediate portions comprise a dual-lead thread, wherein a pitch of the single-lead thread in the distal portion is greater than a pitch of the dual-lead thread in the proximal and intermediate portions.

15. The method of claim 14, wherein the proximal portion has a greater thread cross-section diameter than the intermediate and distal portions.

16. The method of claim 14, wherein the neck portion of the bone screw comprises a frustoconical portion.

17. The method of claim 14, wherein the pitch of the single-lead thread in the distal portion is between 3-6 mm.

18. The method of claim 14, wherein the pitch of the single-lead thread in the distal portion is greater than 4 mm.

19. The method of claim 14, wherein the distal portion of the shank portion is slightly tapered.

20. The method of claim 14, wherein extending the bone screw into the vertebral body comprises extending the bone screw into cancellous bone.
